(19) United States
(12) Patent Application Publication
(10) Pub. No.: US 2025/0248850 A1
(43) Pub. Date: Aug. 7, 2025

(54) OPHTHALMIC SYSTEMS THAT PRESENT INFORMATION DESCRIBING AN EYE

(71) Applicant: Alcon Inc., Fribourg (CH)

(72) Inventors: Stefan Schmid, Neuendettelsau (DE); Andreas Vetter, Erlangen (DE)

(21) Appl. No.: 19/032,573

(22) Filed: Jan. 21, 2025

Related U.S. Application Data
(60) Provisional application No. 63/548,638, filed on Feb. 1, 2024.

(51) Int. Cl. A61F 9/008; A61F 2/16

(52) U.S. Cl. CPC A61F 9/008; A61F 2/16; A61F 2009/00844; A61F 2009/0087; A61F 2009/00878

(57) ABSTRACT

In certain embodiments, an ophthalmic system that presents information describing an eye includes a memory, display device, and computer. The memory stores data describing the eye. The data includes measurements of the eye, and the measurements include one or more eye model measurements used to generate an eye model of the eye. The display device presents an image. The computer applies a light propagation technique to the eye model measurements to generate the eye model of the eye. The computer receives a selection of one or more parameters to use in a simulation performed using the eye model and applies the selected parameters to the eye model to yield one or more simulated focal areas. The computer instructs the display device to present the image that includes the simulated focal areas relative to the retinal plane of the eye.

### TECHNICAL FIELD

The present disclosure relates generally to ophthalmic systems, and more particularly to ophthalmic systems that present information describing an eye.

### BACKGROUND

Ophthalmic diagnostic devices can provide a wide variety of diagnostic data that describe an eye. Diagnostic devices include slit lamp, optical coherence tomography (OCT), ultrasound, aberrometer, topography, and other devices. These devices provide diagnostic data such as measurements of the dimensions (e.g., length) and surface shapes (e.g., anterior corneal surface shape) of the eye.

Eye measurements are important for diagnosing and treating an eye. For example, accurate eye measurements are needed to select the appropriate intraocular lens (IOL) or to design an effective laser surgical procedure to correct vision. However, understanding and utilizing large amounts of diagnostic data can be difficult.

### BRIEF SUMMARY

In certain embodiments, an ophthalmic system that presents information describing an eye includes a memory, display device, and computer. The memory stores data describing the eye. The data includes measurements of the eye, and the measurements include one or more eye model measurements used to generate an eye model of the eye. The display device presents an image. The computer applies a light propagation technique to the eye model measurements to generate the eye model of the eye. The computer receives a selection of one or more parameters to use in a simulation performed using the eye model and applies the selected parameters to the eye model to yield one or more simulated focal areas. The computer instructs the display device to present the image that shows the simulated focal areas relative to the retinal plane of the eye.

Embodiments may include none, one, some, or all of the following features:
The selected parameters include a trial measurement of the eye, and the image shows the simulated focal areas resulting from the trial measurement relative to an accepted focal area.
The selected parameters include a refractive correction for the eye, and the image shows the simulated focal areas resulting from the refractive correction for the eye.
The selected parameters include an intraocular lens (IOL) correction for the eye, and the image shows the simulated focal areas resulting from the IOL correction for the eye.
The selected parameters include a contact lens correction for the eye, and the image shows the simulated focal areas resulting from the contact lens correction for the eye.
The selected parameters include a pre-operation measurement and a post-operation measurement, and the image shows a simulated pre-operation focal area resulting from the pre-operation measurement and a simulated post-operation focal area resulting from the post-operation measurement.
The selected parameters include a first set of one or more measurements and a second set of one or more measurements, and the image shows a first simulated focal area resulting from the first set of one or more measurements and a second simulated area resulting from the second set of one or more measurements.
The image represents a simulated focal area as light rays intersecting at the simulated focal area.
The image represents a simulated focal area as a frequency distribution of intensity at the simulated focal area.
The image represents a simulated focal area as a depth of focus of the simulated focal area.
The image comprises one or more graphical elements configured to receive a user interaction indicating the selection of one or more parameters.
The computer receives post-operation feedback regarding the eye model and adjusts the eye model in response to the post-operation feedback.
The computer checks whether the selected parameters are within an accepted range and provides a notification if a parameter is not within the accepted range.
The light propagation technique comprises ray tracing.
The light propagation technique comprises physical optics propagation.
The computer receives a selection of one or more axes of the eye. Each simulated focal area is a focal area of an axis of the selected axes. The axes may include a flat axis, steep axis, and/or user-defined axis.
The ophthalmic system includes a laser surgical device that uses information from the simulation to perform an ophthalmic operation. The computer may send the information from the simulation to the laser surgical device to perform the ophthalmic operation.

### BRIEF DESCRIPTION OF THE DRAWINGS

FIG. 1 illustrates an example of a system for presenting information describing an eye, according to certain embodiments;

FIG. 2 illustrates an example of an image with information describing an eye that may be presented by system of FIG. 1, according to certain embodiments;

FIGS. 3A to 3D illustrate examples of representations of focal areas that may be included in the image of FIG. 2, according to certain embodiments;

FIGS. 4A to 4D illustrate examples of selectors that may be included in the image of FIG. 2, according to certain embodiments;

FIGS. 5A to 5C illustrate examples of types of focal areas that may be included in the image of FIG. 2, according to certain embodiments; and

FIG. 6 illustrates an example of a method for presenting eye information that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Large amounts of diagnostic data can be difficult to understand and utilize. In current ophthalmic systems, the user must check the plausibility of the data by comparing large numbers of images and values, which may be overwhelming. Moreover, some data are expressed in ways that are difficult to interpret, understand, or compare (e.g., as Zernike values).

Certain embodiments of the systems described in the present disclosure synthesize diagnostic data of an eye into a coherent eye model that can be used to present information describing the eye. In the embodiments, ray tracing and/or physical optics propagation is applied to the diagnostic data to create the eye model. The eye model can then be used to simulate the effects of different model parameters to yield eye information (e.g., focal points and/or point spread functions) that can be displayed to a user. For example, the eye model can be used to evaluate a set of measurements by comparing a focal point resulting from the measurements to an accepted reference focal point. As another example, the eye model can be used to simulate the effects of a proposed refractive correction.

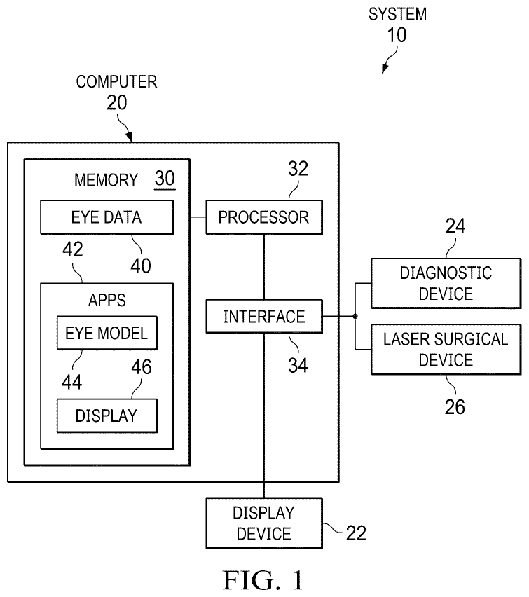

FIG. 1 illustrates an example of a system 10 for presenting information describing an eye, according to certain embodiments. In the example, system 10 includes a computer 20, a display device 22, a diagnostic device 24, and a laser surgical device 26, coupled as shown. Computer 20 includes a memory 30, a processor 32, and an interface (IF) 34. Memory 30 stores eye data 40 and applications (apps) 42, which include eye model 44 and display 46 applications.

In an example overview, memory 30 stores eye data 40, which includes measurements of the eye including, e.g., eye model measurements used to generate an eye model of the eye. Computer 20 applies a light propagation technique (e.g., ray tracing and/or physical optics propagation) to the eye model measurements to generate the eye model. Computer 20 receives selected parameters to use in a simulation with the eye model, applies the selected parameters to the eye model to yield a simulated focal area, and instructs display device 22 to present an image of the simulated focal area relative to the retinal plane of the eye.

Turning to the components of system 10, display device 22 presents an image according to image data provided by computer 20. The image may be a two- (2D) or three-dimensional (3D) image. Display device 22 may be, e.g., a computer monitor, television screen, system display, near-to-eye display panel (e.g., in glasses such as 3D or virtual/augmented reality glasses), oculars (e.g., digital microscope oculars), or other suitable device that can present an image as a mechanical or electronic display.

Diagnostic device 24 measures the eye to yield eye measurements, which can be added to eye data 40. Diagnostic device 24 may be any suitable device that can provide measurements of an eye. Examples of diagnostic devices 24 include: (1) aberrometers such as wavefront aberrometers that measure the optical behavior of the eye; (2) biometers that measure the dimensions of the eye, e.g., axial length, anterior chamber depth, and lens thickness; (3) tomographers (e.g., Placido, Scheimpflug, or OCT tomographer) that measure the shape and/or tissue structure of the cornea and/or lens.

In certain embodiments, system 10 includes laser surgical device 26, which may perform an ophthalmic operation, such as refractive surgery (e.g., Laser-Assisted In Situ Keratomileusis (LASIK), Laser-Assisted Sub-Epithelial Keratomileusis (LASEK), Photo-Refractive Keratectomy (PRK), Small Incision Lenticule Extraction (SMILE), or conductive keratoplasty), lens replacement or implantation surgery (e.g., intraocular lens (IOL), phakic IOL, or refractive lens exchange), or other suitable procedure on an eye. In the embodiments, laser surgical device 26 may use information from the simulation to perform the ophthalmic operation. For example, computer 20 may send the simulation information to laser surgical device 26, and/or computer 20 may utilize the simulation information to generate instructions to laser surgical device 26 to perform the ophthalmic operation.

Eye data 40 is data describing an eye, such as measurements of the eye and data collected from a population of eyes. In certain embodiments, eye data 40 may include a standard eye model, such as a Gullstrand, Le-Grand, Emsley, Navarro, and or other eye model based on, e.g., an average population. Computer 20 may use a standard eye model as a starting point to generate an eye model for a patient's eye. In certain embodiments, eye data 40 may include accepted measurements. Certain eye measurements may be regarded as more reliable and thus designated as accepted measurements. Examples of accepted measurements include manifest refraction, axial length, and cycloplegic measurements, and in some situations the curvature of the eye and eye dimensions (e.g., eye length, corneal thickness, anterior chamber depth, and lens thickness).

Computer 20 controls components of system 10 (e.g., display device 22, diagnostic device 24, and/or laser surgical device 26) to present eye information. In certain embodiments, computer 20 generates an eye model for the patient's eye. In the embodiments, computer 20 may apply a light propagation technique to eye data 40 (e.g., eye measurements and/or a standard eye model) to generate the model. Light propagation techniques describe how light passes through an optical system, e.g., the eye. Examples of such techniques include geometrical optics (e.g., ray propagation such as ray tracing), physical optics (e.g., wavefront propagation), or other suitable techniques that describe how light passes through an optical system. The ray tracing technique propagates light rays forward and/or backward through the eye, e.g., through the cornea, aqueous humor, lens, and vitreous humor to the retina, as described by eye data 40. The ray tracing shows light rays refracted by the optical surfaces and the resulting focal points. In a similar manner, the physical optics propagation technique propagates wavefronts, which may be represented by an array of discretely sampled points. In certain embodiments, the eye measurements of the patient's eye may take precedence over the standard eye model so the resulting eye model may more accurately represent the patient's eye.

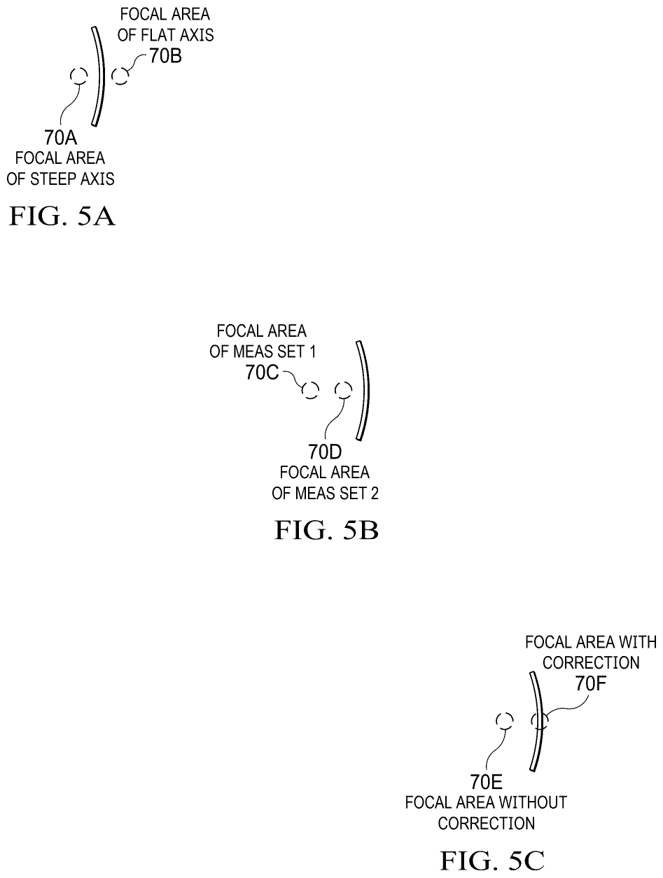

Computer 20 applies selected parameters to the eye model to yield simulated focal areas resulting from the selected parameters and instructs display device 22 to display the simulated focal areas relative to the retinal plane of the eye. Any suitable parameter may be used for the simulation. For example, a measurement may be tested for plausibility (a “trial measurement”). In the example, the trial measurement is used in the eye model to generate a simulated focal area, which is compared to a focal area generated from accepted eye measurements. As another example, a proposed correction (e.g., a refractive or IOL correction) may be used in the eye model to generate a simulated focal area resulting from the correction. As another example, measurements may be taken before and after an ophthalmic operation, such as refractive surgery (e.g., LASIK, LASEK, PRK, SMILE, or conductive keratoplasty) or a lens replacement/implantation procedure (e.g., IOL, phakic IOL, or refractive lens exchange). The pre-operation and post-operation measurements are used in the model to generate simulated pre-operation and post-operation focal areas, respectively, resulting from the measurements. Examples are described with reference to FIGS. 5A to 5C.

Computer 20 may perform any other suitable operations. In certain embodiments, computer 20 checks whether the selected parameters or measurements are within an accepted range and provides a notification if they are not within the accepted range. The accepted range may be determined from a standard eye model or from accepted measurements of the eye.

In certain embodiments, computer 20 may receive post-operation feedback about the eye model and apply machine learning to improve the eye model. For example, post-operation feedback may include measurements indicating the actual post-operation focal area. Computer 20 compares the simulated and actual post-operation focal areas. If there is a large discrepancy related to a particular factor, e.g., a higher diopter refractive correction, computer 20 may adjust the assumptions related to higher diopters.

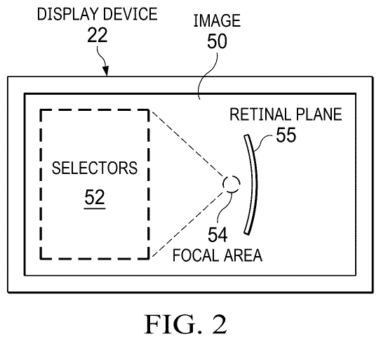

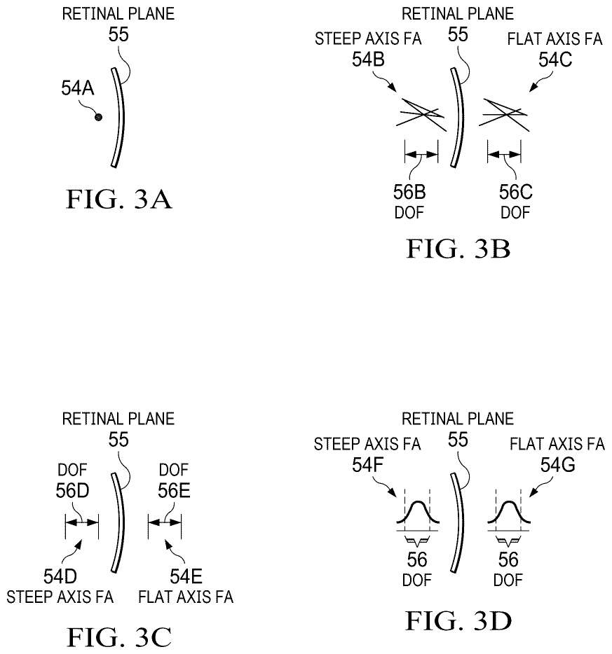

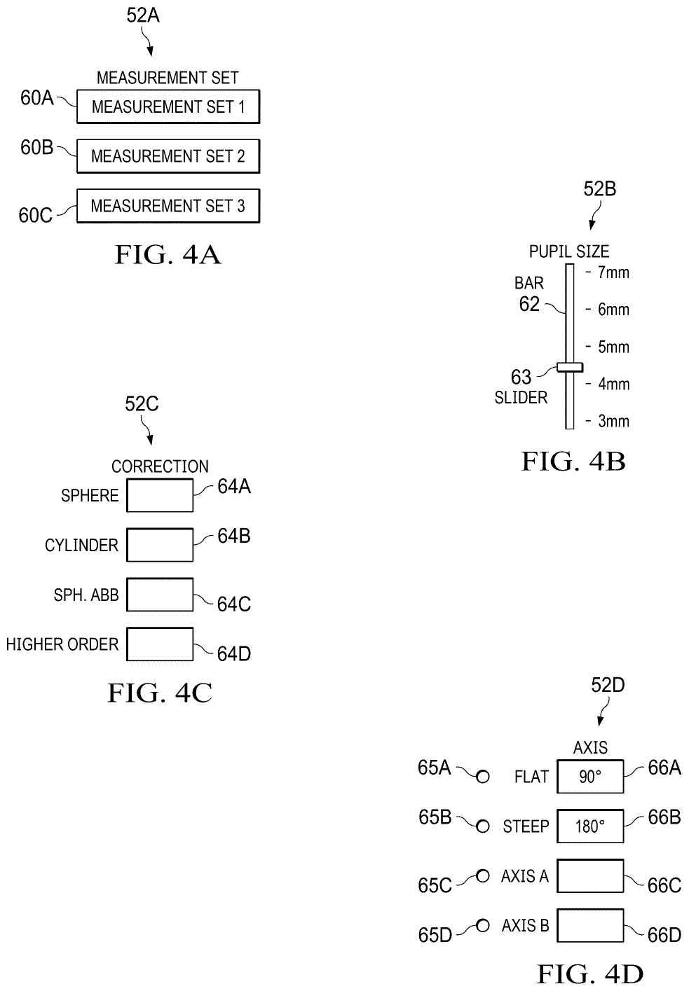

FIG. 2 illustrates an example of an image 50 with information describing an eye that may be presented by system 10 of FIG. 1, according to certain embodiments. Image 50 includes selectors 52 and a focal area 54 shown relative to the retinal plane 55. Selectors 52 may be used to select parameters to apply to the eye model to determine focal area 54. Examples of selectors are described with respect to FIGS. 3A to 3D. Examples of representations of focal areas are described with respect to FIGS. 4A to 4D. Examples of types of focal areas are described with respect to FIGS. 5A to 5C.

FIGS. 3A to 3D illustrate examples of representations of focal areas 54 (54a to 54g) that may be included in the image of FIG. 2, according to certain embodiments. FIG. 3A shows the focal area 54 (FA) of an example myopic eye as a dot representing the focal point relative to the retinal plane 55. FIGS. 3B to 3D show the steep and flat axis focal areas 54 of an example astigmatic eye. Astigmatic eyes are often characterized by one axis that is relatively steep and another axis that is relatively flat, which can lead to multiple focal points. A focal area 54 may include the focal point and/or the depth of focus (DOF) around the focal point. In the examples, focal areas 54b, 54d, 54f represent the focal area of the steep axis, and focal areas 54c, 54c, 54g represent the focal area of the flat axis. In other examples, the focal areas of any other suitable axes may be shown, e.g., a user-defined axis.

FIG. 3B shows focal areas 54b and 54c as the intersection of light rays converging at the focal area 54b and 54c, respectively. Focal area 54b represents the focal area of the steep axis, and focal area 54c represents the focal area of the flat axis. The representation also includes an indicator of the depths of focus (DOF) 56b and 56c of the steep axis focal area 54b and the flat axis focal area 54c, respectively.

FIG. 3C shows focal areas 54d and 54e as DOFs 56d and 56e, respectively, of the steep axis focal area 54d and the flat axis focal area 54e, respectively. FIG. 3D shows focal areas 54 (54f and 54g) as the frequency distribution of intensity of or point spread function across the DOFs 56f and 56g, respectively, of the steep axis focal area 54f, and the flat axis focal area 54g, respectively.

FIGS. 4A to 4D illustrate examples of selectors 52 (52a to 52c) that may be included in the image of FIG. 2, according to certain embodiments. A selector 52 is a graphical element that allows the user to select parameters for the simulation. Examples of graphical elements include buttons, menus, dials, fields, or other graphical elements that can receive a user interaction to make a selection.

FIG. 4A shows a measurement set selector 52a. Selector 52a includes graphical elements, e.g., buttons 60 (60a to 60c), that each represent a measurement set that can be used for parameters for an eye model. In the example, buttons 60a, 60b, and 60c can be selected to select Measurement Sets 1, 2, and 3, respectively.

FIG. 4B shows a pupil size selector 52b. Selector 52b includes a graphical element that may be used to select the pupil size. In the example, a bar 62 extends across a range of pupil sizes (3 millimeters (mm) to 7 mm in the example), and a slider 63 can be moved across bar 62 to select a size.

FIG. 4C shows a correction selector 52c. Selector 52c includes graphical elements that may be used to select correction parameters. In the example, the elements include fields 64 such as fields 64a, 64b, 64c, and 64d into which parameters for the sphere, cylinder, spherical aberration, and higher order variables, respectively, can be entered.

FIG. 4D shows an axis selector 52d. Selector 52d includes graphical elements that may be used to select the axes for which the focal areas can be displayed. Any suitable axis or axes can be selected, such as the flat axis, the steep axis, and/or a user-defined axis other than the flat or steep axis. In the example, the graphical elements include buttons 65 such as buttons 65a, 65b, 65c, and 65d that can be used to select Flat Axis, Steep Axis, Axis A, and Axis B, respectively. Fields 66 may be used to enter and/or display the values for the axes. For example, fields 66a, 66b, 66c, and 66d may be used for Flat Axis, Steep Axis, Axis A, and Axis B, respectively. Continuing the example, a user may use fields 66c and/or 66d to enter user-defined custom values for Axis A and/or Axis B, respectively.

FIGS. 5A to 5C illustrate examples of types of focal areas 70 (70a to 70f) that may be included in the image of FIG. 2, according to certain embodiments. FIG. 5A shows focal areas of the steep axis 70a and flat axis 70b of an example of a mixed astigmatic eye. In other examples, the focal areas of any other suitable axis may be shown, e.g., a user-defined axis.

FIG. 5B shows focal areas of different measurement sets, i.e., focal area 70c of measurement set M1 and focal area 70d of measurement set M2. A measurement set may include one or more measurements, which may be measurements of the patient's eye, accepted measurements, and/or standard measurements. The measurement sets may be used for any suitable purpose. For example, a measurement of the patient's eye may be tested for plausibility (a “trial measurement”). In the example, measurement set M1 includes the trial measurement and is used to generate a simulated focal area, which is compared to an accepted focal area generated from a measurement set M2 that includes an accepted eye measurement. If the simulated focal area does not match the accepted focal area, computer 20 may provide a notification that the trial measurement should be checked by the user.

As another example, measurement set M1 includes pre-operation measurements, and measurement set M2 includes post-operation measurements, which are used to generate simulated pre-operation and post-operation focal areas. In certain cases, the pre-operation and post-operation simulations can be compared to detect surgical factors that impact vision, e.g., epithelial wound healing effects. Computer 20 may then incorporate the information into diagnostic data 40 used to generate future eye models.

FIG. 5C shows focal areas 70e and 70f without correction and with correction, respectively. In an example, a proposed correction (e.g., a refractive, intraocular lens (IOL), or contact lens correction) may be used in the eye model as parameters to generate a simulated focal area resulting from the correction. A refractive correction may be expressed in the eye model as a modified curvature of the corneal surface, and an IOL or contact lens correction may be expressed as an added layer in the eye model. Display device 22 shows the simulated focal area resulting from the refractive correction.

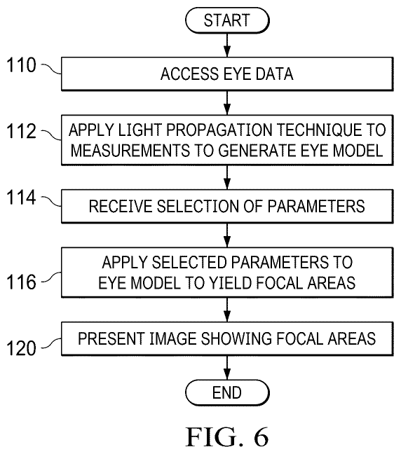

FIG. 6 illustrates an example of a method for presenting eye information that may be performed by system 10 of FIG. 1, according to certain embodiments. The method starts at step 110, where computer 110 accesses eye data 40 in memory 30. Eye data 40 may include measurements of the eye, standard eye models, accepted measurements, and/or other descriptions of the patient's eye and/or a population of eyes.

Computer 20 applies a light propagation technique (e.g., ray tracing and/or physical optics propagation) at step 112 to eye model measurements to generate an eye model of the eye. Computer 20 may apply the light propagation technique to, e.g., eye measurements and/or a standard eye model, to generate the eye model. A selection of parameters is received from the user at step 114. Example parameters include a trial measurement for simulation, a proposed eye correction, measurement sets, or pre-operation and post-operation measurements.

Computer 20 applies the selected parameters at step 116 to the eye model to yield one or more simulated focal areas. The simulated focal areas may be used, e.g., to evaluate a trial measurement or proposed correction or to compare measurement sets or pre-operation and post-operation measurements. At step 120, computer 20 instructs display device 22 to present the image showing the simulated focal areas relative to the retinal plane of the eye.

A component (such as the control computer 20) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic system that presents information describing an eye, comprising:
a memory configured to store data describing the eye, the data comprising a plurality of measurements of the eye, the measurements comprising one or more eye model measurements used to generate an eye model of the eye;
a display device configured to present an image; and
a computer configured to:
apply a light propagation technique to the one or more eye model measurements to generate the eye model of the eye;
receive a selection of one or more parameters to use in a simulation performed using the eye model;
apply the selected one or more parameters to the eye model to yield one or more simulated focal areas; and
instruct the display device to present the image comprising the one or more simulated focal areas relative to a retinal plane of the eye.

2. The ophthalmic system of claim 1, wherein:
the selected one or more parameters comprise a trial measurement of the eye; and
the image comprises the one or more simulated focal areas resulting from the trial measurement relative to an accepted focal area.

3. The ophthalmic system of claim 1, wherein:
the selected one or more parameters comprise a refractive correction for the eye; and
the image comprises the one or more simulated focal areas resulting from the refractive correction for the eye.

4. The ophthalmic system of claim 1, wherein:
the selected one or more parameters comprise an intraocular lens (IOL) correction for the eye; and
the image comprises the one or more simulated focal areas resulting from the IOL correction for the eye.

5. The ophthalmic system of claim 1, wherein:
the selected one or more parameters comprise a contact lens correction for the eye; and
the image comprises the one or more simulated focal areas resulting from the contact lens correction for the eye.

6. The ophthalmic system of claim 1, wherein:
the selected one or more parameters comprise a pre-operation measurement and a post-operation measurement; and
the image comprises:
a simulated pre-operation focal area resulting from the pre-operation measurement; and
a simulated post-operation focal area resulting from the post-operation measurement.

7. The ophthalmic system of claim 1, wherein:
the selected one or more parameters comprise a first set of one or more measurements and a second set of one or more measurements; and
the image comprises:
a first simulated focal area resulting from the first set of one or more measurements; and
a second simulated area resulting from the second set of one or more measurements.

8. The ophthalmic system of claim 1, wherein the image represents a simulated focal area of the one or more simulated focal areas as a plurality of light rays intersecting at the simulated focal area.

9. The ophthalmic system of claim 1, wherein the image represents a simulated focal area of the one or more simulated focal areas as a frequency distribution of intensity at the simulated focal area.

10. The ophthalmic system of claim 1, wherein the image represents a simulated focal area of the one or more simulated focal areas as a depth of focus of the simulated focal area.

11. The ophthalmic system of claim 1, wherein the image comprises:
one or more graphical elements configured to receive a user interaction indicating the selection of the one or more parameters.

12. The ophthalmic system of claim 1, the computer configured to:
receive post-operation feedback regarding the eye model; and
adjust the eye model in response to the post-operation feedback.

13. The ophthalmic system of claim 1, the computer configured to:
check whether the selected one or more parameters are within an accepted range; and
provide a notification if a parameter of the selected one or more parameters is not within the accepted range.

14. The ophthalmic system of claim 1, wherein the light propagation technique comprises ray tracing.

15. The ophthalmic system of claim 1, wherein the light propagation technique comprises physical optics propagation.

16. The ophthalmic system of claim 1, wherein:
the computer is configured to receive a selection of one or more axes of the eye; and
for each simulated focal area of the one or more simulated focal areas, the each simulated focal area is a focal area of an axis of the one or more axes of the eye.

17. The ophthalmic system of claim 16, wherein:
the one or more axes comprise a flat axis, a steep axis, or both.

18. The ophthalmic system of claim 16, wherein:
the one or more axes comprise a user-defined axis.

19. The ophthalmic system of claim 1, further comprising a laser surgical device configured to use information from the simulation to perform an ophthalmic operation.

20. The ophthalmic system of claim 19, wherein the computer is configured to send the information from the simulation to the laser surgical device to perform the ophthalmic operation.
